SEQUENCE LISTING (51 sequences)

SEQ ID NO: 1 - protein, 119 aa, Human [sequence omitted]

SEQ ID NO: 2 - DNA, 35 bp, Artificial
  Primer 104025
cctgaattcacgcgcgccaacatgtcttccaagtc

SEQ ID NO: 3 - DNA, 31 bp, Artificial
  Primer 104026
gttctcgagctacttattgcgcaccaacacg

SEQ ID NO: 4 - DNA, 18 bp, Artificial
  Primer 104027
accatggcggcactctgc

SEQ ID NO: 5 - DNA, 18 bp, Artificial
  Primer  104028
gagccgtaggggaagtcc

SEQ ID NO: 6 - DNA, 19 bp, Artificial
  Primer 108089
cttcagactgaacctcgcc

SEQ ID NO: 7 - DNA, 20 bp, Artificial
  Primer 108091
gactcggtccgtacattgcc

SEQ ID NO: 8 - DNA, 38 bp, Artificial
  Primer
cctacggctccgagagaggccttttgatccttgcggag

SEQ ID NO: 9 - DNA, 20 bp, Artificial
  Primer 135944
gagttagtagttggacatcc

SEQ ID NO: 10 - DNA, 30 bp, Artificial
  Primer 141200J1
atcggttttatgtcttccaagtcgcaattg

SEQ ID NO: 11 - DNA, 33 bp, Artificial
  Primer 141200J2
cttggaagacataaaaccgatggaggggtagcg

SEQ ID NO: 12 - DNA, 26 bp, Artificial
  Primer 270999J8
tctgtgaggcctatggatctcagaac

SEQ ID NO: 13 - DNA, 27 bp, Artificial
  Primer 270999J9
gatgctgcatgcacaactgcacctcag

SEQ ID NO: 14 - DNA, 59 bp, Artificial
  Primer 051199J1
cctctagatctcgagctcggtcaccggtggcctccgcggccgctggatccccagttgtg

SEQ ID NO: 15 - DNA, 33 bp, Artificial
  Primer 1298TAKA
gcaagcgcgcgcaatacatggtgttttgatcat

SEQ ID NO: 16 - DNA, 31 bp, Artificial
  Primer 142779
ttgaattgaaaatagattgatttaaaacttc

SEQ ID NO: 17 - DNA, 25 bp, Artificial
  Primer 142780
ttgcatgcgtaatcatggtcatagc

SEQ ID NO: 18 - DNA, 26 bp, Artificial
  Primer 14288
ttgaattcatgggtaataactgatat

SEQ ID NO: 19 - DNA, 32 bp, Artificial
  Primer 142778
aaatcaatctattttcaattcaattcatcatt

SEQ ID NO: 20 - DNA, 45 bp, Artificial
  Primer 141223
ggatgctgttgactccggaaatttaacggtttggtcttgcatccc

SEQ ID NO: 21 - DNA, 44 bp, Artificial
  primer 141222
ggtattgtcctgcagacggcaatttaacggcttctgcgaatcgc

SEQ ID NO: 22 - DNA, 27 bp, Artificial
  Primer P50 - 260301j1
acaggactcggtncgtacattgccgtg

SEQ ID NO: 23 - DNA, 27 bp, Artificial
  Primer F91 - 260301j2
aatttcctcatctncgaagatcgcaag

SEQ ID NO: 24 - DNA, 27 bp, Artificial
  Prime F96 - 260301j3
gaagatcgcaagtncatcgatatcgga

SEQ ID NO: 25 - DNA, 30 bp, Artificial
  Primer N101,T102 - 260301j4
atcgatatcgganacancgtccaaaagcag

SEQ ID NO: 26 - DNA, 27 bp, Artificial
  Primer G128 - 260301j5
agtattctgcccgntgagggtatcgtc

SEQ ID NO: 27 - DNA, 30 bp, Artificial
  Primer G222, D223 - 260301j6
ctctcctcgaaggntnacaagctgggacag

SEQ ID NO: 28 - DNA, 27 bp, Artificial
  Primer G239 - 230301j7
gctgttggacgcgntgccgactttatt

SEQ ID NO: 29 - DNA, 69 bp, Artificial
  Primer 210201J1
gcctctagatctcccgggcgcgccggcacatgtaccaggtcttaagctcgagctcggtcaccggtggcc

SEQ ID NO: 30 - DNA, 59 bp, Artificial
  Primer 230402j3
accttcaccgactacacgatggactgggtccggcaggcgccgggcaagggcctggagtg

SEQ ID NO: 31 - DNA, 70 bp, Artificial
  Primer 230402j4
ccgggcaagggcctggagtgggtcgcggacgtgaacccgaactccggcgggtcgatctacaaccagcgct

SEQ ID NO: 32 - DNA, 60 bp, Artificial
  Primer 230402j7
agacggcggtgtcctccgcccggagggagttcatctgcaggtacagcgtgttcttcgacc

SEQ ID NO: 33 - DNA, 70 bp, Artificial
  Primer 230402j8
gtacagcgtgttcttcgaccggtcgaccgagagcgtgaaccggcccttgaagcgctggttgtagatcgac

SEQ ID NO: 34 - DNA, 60 bp, Artificial
  Primer 230402J1
gaggtccagctcgtcgagtccggcggcggcctcgtgcagccggggggctcgctgcggctc

SEQ ID NO: 35 - DNA, 60 bp, Artificial
  Primer 230402j2
cggggggctcgctgcggctctcctgcgccgcgtcgggcttcaccttcaccgactacacga

SEQ ID NO: 36 - DNA, 72 bp, Artificial
  Primer 230402j5
atcgagccgcggctacgaggagacggtgaccagggtgccctggccccagtagtcgaagtagaacgacgggcc

SEQ ID NO: 37 - DNA, 60 bp, Artificial
  Primer 230402j6
tcgaagtagaacgacgggccgaggttccgggcgcagtagtagacggcggtgtcctccgcc

SEQ ID NO: 38 - DNA, 41 bp, Artificial
  Primer 230402J9
gactcgacgagctggacctccgagccagggcacgcggacgg

SEQ ID NO: 39 - DNA, 38 bp, Artificial
  Primer 090103j1
gtagacggatccaccatgaaggcgatcctctctctcgc

SEQ ID NO: 40 - DNA, 45 bp, Artificial
  Primer 081102J5
gccttggctagccctattcgtcgagaggtccagctcgtcgagtcc

SEQ ID NO: 41 - DNA, 27 bp, Artificial
  Primer 211102j1
cacgagctcgagccgcggctacgagga

SEQ ID NO: 42 - DNA, 45 bp, Artificial; identical to SEQ ID NO: 40
  Primer 081102J5

SEQ ID NO: 43 - DNA, 36 bp, Artificial
  Primer 030103j1
gtcagcgctagccgaggagacggtgaccagggtgcc

SEQ ID NO: 44 - DNA, 66 bp, Artificial
  Primer 991213j5
cctctsgatctcgagctcggtcaccggtggcctccgcggccgctgcgccaggtgtcagtcaccctc

SEQ ID NO: 45 - DNA, 33 bp, Artificial
  Primer 301202j1 V37X, Q39X
acgatggactggnnscggnnsgcgccgggcaag

SEQ ID NO: 46 - DNA, 36 bp, Artificial
  Primer 301202j2 G44X, L45X, W47X
gcgccgggcaagnnsnnsgagnnsgtcgcggacgtg

SEQ ID NO: 47 - DNA, 27 bp, Artificial
  Primer 301202j3 Y95 X
accgcggtctacnnstgcgcccggaac

SEQ ID NO: 48 - DNA, 26 bp, Artificial
  Primer 301202j4 W109X
acttcgactacnnsggccagggcacc

SEQ ID NO: 49 - DNA, 30 bp, Artificial
  Primer 7887
gaatgacttggttgagtactcaccagtcac

SEQ ID NO: 50 - DNA, 21 bp, Artificial
  Primer 19670
ccccatcctttaactatagcg

SEQ ID NO: 51 - DNA, 22 bp, Artificial
  Primer 060302J1
agagcttaaagtatgtcccttg

(19) United States
(12) Patent Application Publication
(10) Pub. No.: US 2006/0234340 A1
(43) Pub. Date: Oct. 19, 2006

(54) Human heavy chain antibody expression in flamentous fungi

(75) Inventor: Jesper Vind, Vaerlose (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/544,302

(22) PCT Filed: Feb. 6, 2004
(86) PCT No.: PCT/DK04/00086

§ 371 Date: Jun. 15, 2006

(30) Foreign Application Priority Data
Feb. 6, 2003 (DK) PA 2003 00169

(51) Int. Cl. C12P 21/06; C07H 21/04; C07K 16/44; C12N 1/16; C12N 15/74

(52) U.S. Cl. 435/69.1; 530/388.15; 435/254.3; 435/484; 536/23.53

(57) ABSTRACT

The present invention relates to a method for producing a functional human immunoglobulin, wherein a human heavy chain immunoglobulin, devoid of any light chain, is expressed, comprising the steps of: a) transforming a filamentous host cell with a recombinant construct encoding a modified human heavy chain immunoglobulin, wherein the modifications comprise one or more mutations in the region of the heavy chain protein involved in contact with the light chain; b) culturing said filamentous host cell under conditions promoting expression of said modified human heavy chain immunoglobulin; and c) recovering said modified human heavy chain immunoglobulin.

### FIELD OF INVENTION

The present invention relates to expression of human immunoglobulin heavy chain proteins and fragments thereof in filamentous fungi.

### BACKGROUND OF THE INVENTION

For decades there has been a focus on the use of antibodies for therapeutics. At the same time there has also been a lot of focus on the production of antibodies. Today the expression of therapeutic antibodies takes place in mammalian cells, which is difficult and expensive. Many attempts have been done to express antibodies in microbial organisms because they have a large expression potential and are easy to handle.

Expression of antibodies in these organisms, however, has turned out to be difficult, especially because antibodies consist of two proteins (heavy and light chain). Recently it was discovered that the Camelidae family express an antibody type, which only consists of the heavy-chain protein. None the less, this type of antibody can have the same degree of affinity as normal antibodies. This is because the variable domain on the heavy-chain is larger. It has turned out that some of these antibodies can be expressed in a yeast or in a mould, see e.g WO 94/25591.

The heavy-chain protein of the Camelidae family is quite homologous to the human heavy chain protein, except one of the variable regions being somewhat larger.

In order to solve the problems of efficient expression of human antibodies in non-mammalian expression systems we have looked for other suitable organisms in which expression of human antibody is possible resulting in functional antibodies comprising only a modified heavy chain.

### SUMMARY OF THE INVENTION

Surprisingly we have discovered that it is possible to obtain functional human antibodies or fragments of human antibodies by expression of only the heavy chain of the human antibody in filamentous fungi, such as Aspergillus. Furthermore functional modified heavy chain human antibodies or fragments of the human heavy chain protein can be efficiently expressed in filamentous fungi, such as Aspergillus, and the problem of the low solubility of the human heavy chain protein can be solved by introducing the appropriate mutations in the region that is usually in contact with the light chain.

The present invention relates to a method for producing a functional human immunoglobulin, wherein a human heavy chain immunoglobulin, devoid of any light chain, is expressed, comprising the steps of:
a) transforming a filamentous host cell with a recombinant construct encoding a modified human heavy chain immunoglobulin, wherein the modifications comprises one or more mutations in the region of the heavy chain protein involved in contact with the light chain;
b) culturing said filamentous host cell under conditions promoting expression of said modified human heavy chain immunoglobulin; and
c) recovering said modified human heavy chain immunoglobulin.
   Definitions

Prior to a discussion of the detailed embodiments of the invention, a definition of specific terms related to the main aspects of the invention is provided.

Functional immunoglobulin: The term “functional immunoglobulin” is defined as an immunoglobulin, which despite only comprising the heavy chain protein or a part thereof, has preserved its functionality in terms of being able to bind to the target antigen, and/or be able to activate the immune system.

Modified immunoglobulin: The term “modified immunoglobulin” is defined as an immunoglobulin wherein one or more amino acids have been substituted, deleted or added/inserted. Particularly the modifications comprise amino acids which in the normal human immunoglobulin are involved in contact between the heavy and the light chain, which contact is believed to affect the solubility of the immunoglobulin. In another embodiment the modifications comprise amino acids which in the normal human immunoglobulin are involved in contact between the heavy chain and the antigen, which modifications affect the specificity of the immunoglobulin.

Functional equivalent: The term “functional equivalent residues” is defined as amino acid residues involved in contact between the heavy and the light chain of the immunoglobulin in question.

Mutations: The term “mutation” is defined as substitutions, deletions or insertions.

### DETAILED DESCRIPTION OF THE INVENTION

It is an object of the present invention to provide a method for the efficient production of a modified human antibody in a filamentous fungus in which method only the heavy chain protein is expressed and the antibody still remain functionally active.

In one embodiment of the invention a modified human heavy chain immunoglobulin or a fragment thereof, which e.g. could be the variable region of the heavy chain protein, is produced by inserting the DNA sequence encoding the modified immunoglobulin in a suitable expression vector and introducing said recombinant vector in a filamentous fungus host cell. The filamentous fungus host cell is then cultured under conditions promoting expression of the human immunoglobulin heavy chain. Subsequently the resulting human immunoglobulin can be recovered and purified applying methods well known in the art.

In one particular embodiment the human heavy chain immunoglobulin or the modified human heavy chain immunoglobulin comprises at least the variable region and the Fc-region recognised by the Fc receptor.

In a further embodiment the human heavy chain immunoglobulin or the modified human heavy chain immunoglobulin comprises at least the variable region.

In a further embbdiment the variable region comprises the peptide sequence shown in SEQ ID NO 1.

In a further embodiment the variable region consists of the peptide sequence shown in SEQ ID NO 1.

The modifications introduced into the modified human heavy chain immunoglobulin comprise mutations in the region of the heavy chain protein involved in contact with the light chain.

In one particular embodiment the said modifications results in an increase solubility of the modified human heavy chain immunoglobulin. (Reichmann (1996) Journal of molecular Biology v. 259 p. 957-969)

Thus in further embodiments the modifications, of the complete heavy chain variable domain of the human immunoglobulin or a fragment thereof comprising at least the variable region or at least the variable region and the Fc-region, comprises mutations in the region of the heavy chain human immunoglobulin involved in contact with the light chain.

The possible residues involved in the above mentioned contact between the heavy and the light chain in the variable region is exemplified below and in the examples using the heavy chain variable domain of the human immunoglobulin, Herceptin (disclosed in WO 01/15730 A1) and involve the following positions of the peptide sequence shown in SEQ ID No. 1: V37, Q39, G44, L45, W47, Y95 and W109.

Heavy chain variable domain of the human immunoglobulin, Herceptin (SEQ ID NO: 1):

evqlvesggglvqpggslrlscaasgftftdytmdwvrqapgkglewvadvnpnsggsiynqrfkgrftlsvdrskntlylqmnslr

aedtavyycarnlgpsfyfdywgqgtlvtvss.

The peptide sequence shown in SEQ ID NO. 1 consists of the heavy chain variable domain of the human immunoglobulin, Herceptin, but in respect of other human heavy chain variable domains the residues involved in the said contact could have different positions as long as the residues are the functional equivalents.

In a further embodiment of the invention the modifications of the invention comprises mutations in the region of the heavy chain protein involved in contact with the light chain, said mutations comprising mutations in either of the residues V37, Q39, G44, L45, W47, Y95 and W109 in SEQ ID NO 1, said sequence representing the heavy chain variable domain of the human immunoglobulin, Herceptin, or mutations in functionally equivalent residues in other human heavy chain immunoglobulins.

The above mentioned positions can be identified in other heavy chain variable domains by homology search and alignment by means of computer programs known in the art, such as BlastP (BLASTP 2.1.2 (Reference: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database searchprograms”, Nucleic Acids Res. 25:3389-3402.) using default settings or FastaP (version 3.3t08, W. R. Pearson & D. J. Lipman PNAS (1988) 85:2444-2448) using default settings.

Default settings was as indicated below:
blastall arguments:
   -p Program Name [String]
   -d Database [String]
      default=nr
   -i Query File [File In)
      default=stdin
   -e Expectation value (E) [Real]
      default=10.0
   -m alignment view options:
0=pair wise,
1=query-anchored showing identities,
2=query-anchored no identities,
3=flat query-anchored, show identities,
4=flat query-anchored no identities,
5=query-anchored no identities and blunt ends,
6=flat query-anchored, no identities and blunt ends,
7=XML Blast output [Integer]
   default=0
   -o BLAST report Output File [File Out] Optional
      default=stdout
   -F Filter query sequence (DUST with blastn, SEG with others) [String]
      default=T
   -G Cost to open a gap (zero invokes default behavior) [Integer]
      default=0
   -E Cost to extend a gap (zero invokes default behavior) [Integer]
      default=0
   -X X dropoff value for gapped alignment (in bits) (zero invokes default behavior) [Integer]
      default=0
   -l Show Gl's in deflines [T/F]
      default=F
   -q Penalty for a nucleotide mismatch (blastn only) [Integer]
      default=−3
   -r Reward for a nucleotide match (blastn only) [Integer]
      default=1
   -v Number of database sequences to show one-line descriptions for (V) [Integer]
      default=500
   -b Number of database sequence to show alignments for (B) [Integer]
      default=250.
   -f Threshold for extending hits, default if zero [Integer]
      default=0
   -g Perfom gapped alignment (not available with tblastx) [T/F]
      default=T
   -Q Query Genetic code to use [Integer]
      default=1
   -D DB Genetic code (for tblast[nx] only) [Integer]
      default=1
   -a Number of processors to use [Integer]
      default=1
   -O SeqAlign file [File Out] Optional
   -J Believe the query defline [T/F]
      default=F
   -M Matrix [String]
      default=BLOSUM62
   -W Word size, default if zero [Integer]
      default=0
   -z Effective length of the database (use zero for the real size) [Real]
      default=0
   -K Number of best hits from a region to keep (off by default, if used a value of 100 is recommended) [Integer]
      default=0
   -P 0 for multiple hits 1-pass, ₁ for single hit 1-pass, 2 for 2-pass [Integer]
      default=0
   -Y Effective length of the search space (use zero for the real size) [Real]
      default=0
   -S Query strands to search against database (for blast[nx], and tblastx). 3 is both, 1 is top, 2 is bottom [Integer]
      default=3
   -T Produce HTML output [T/F]
      default=F
   -l Restrict search of database to list of Gl's [String] Optional
   -U Use lower case filtering of FASTA sequence [T/F] Optional
      default=F
   -y Dropoff (X) for blast extensions in bits (0.0 invokes default behavior) [Real]
      default=0.0
   -Z X dropoff value for final gapped alignment (in bits) [Integer]
      default=0

The residues and positions given above relating to SEQ ID NO. 1 are the wild type residues.

In still another embodiment the modifications comprise amino acids, which increases the binding specificity and binding affinity to the antigen. Such modifications comprise amino acids which in the normal human immunoglobulin are involved in contact between the heavy chain and the antigen. Said amino acids comprises residues comprised in the positions 27-35, 50-57 and 99-108 in Seq ID No. 1 representing the heavy chain variable domain of the human immunoglobulin, Herceptin, or functionally equivalent positions in other human heavy chain immunoglobolins. These modifications can be identified by standard phage display techniques (Wanderseee N J; Sillah N M; Watkins N A; Scott J P; Ouwehand W H; Hillery C A Blood, Vol. 98 (11 Part 1) pp. 484a (2001l) Azzazy HME; Highsmith Jr W E Clinical Biochemistry, Vol. 35 (6) pp. 425-445 (2002), (165 refs.)), whereby specificity and binding affinity can be tested.

In a still further embodiment the present invention therefore relates to a method according to the invention, wherein the modifications comprises mutations in the region of the human variable heavy chain immunoglobulin involved in contact with the antigen, said mutations comprising mutations in either of the residues comprised in the positions 27- 35, 50-57 and 99-108 in SEQ ID NO 1, said sequence representing the heavy chain variable domain of the human immunoglobulin, Herceptin, or mutations in functionally equivalent residues in other human heavy chain immunoglobulins.

Nucleic Acid Constructs

The present invention also relates to nucleic acid constructs comprising a nucleotide sequence of the present invention operably linked to one or more control sequences that direct the expression of the coding sequence in a suitable host cell under conditions compatible with the control sequences.

A nucleotide sequence encoding a polypeptide of the present invention may be manipulated in a variety of ways to provide for expression of the polypeptide. Manipulation of the nucleotide sequence prior to its insertion into a vector may be desirable or necessary depending on the expression vector. The techniques for modifying nucleotide sequences utilizing recombinant DNA methods are well known in the art.

The control sequence may be an appropriate promoter sequence, a nucleotide sequence which is recognized by a host cell for expression of the nucleotide sequence. The promoter sequence contains transcriptional control sequences, which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Asperqillus niger or Aspergillus awamori glucoamylase (glaA), Rhizomucor miehei lipase, Aspergillus oryzae alkaline proteasce, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

The control sequence may also be a signal peptide-coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choics may be used in the present invention.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the polypeptide would be operably linked with the regulatory sequence.

Expression Vectors

The present invention also relates to recombinant expression vectors comprising the nucleic acid construct of the invention. The various nucleotide and control sequences described above may be joined together to produce a recombinant expression vector which may include one or more convenient restriction sites to allow for insertion or substitution of the nucleotide sequence encoding the polypeptide at such sites. Alternatively, the nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome.

The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof.

Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits stable integration of the vector into the host cell's genome or autonomous replication of the vector in the cell independent of the genome.

For integration into the host cell genome, the vector may rely on the nucleotide sequence encoding the polypeptide or any other element of the vector for stable integration of the vector into the genome by homologous or nonhomologous recombination. Alternatively, the vector may contain additional nucleotide sequences for directing integration by homologous recombination into the genome of the host cell. The additional nucleotide sequences enable the vector to be integrated into the host cell genome at a precise location(s) in the chromosome(s). To increase the likelihood of integration at a precise location, the integrational elements should preferably contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous vwith the target sequence in the genome of the host cell. Furthermore, the integrational elements may be non-encoding or encoding nucleotide sequences. On the other hand, the vector may be integrated into the genome of the host cell by non-homologous recombination.

Host Cells

The present invention also relates to a recombinant host cell comprising the nucleic acid construct of the invention, which are advantageously used in the recombinant production of the polypeptides. A vector comprising a nucleotide sequence of the present invention is introduced into a host cell so that the vector is maintained as a chromosomal integrant or as a self-replicating extra-chromosomal vector as described earlier.

In a preferred embodiment, the host cell is a fungal cell. “Fungi” as used herein includes the phyla Ascomycota, Basidiomycota, Chytridiomycota, and Zygomycota (as defined by Hawksworth et at., In, Ainsworth and Bisby's Dictionary of The Fungi, 8th edition, 1995, CAB International, University Press, Cambridge, UK) as well as the Oomycota (as cited in Hawksworth et at, 1995, supra, page 171) and all mitosporic fungi (Hawksworth et al., 1995, supra).

In another more preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, or Trichoderma.

In a most preferred embodiment, the filamentous fungal host cell is an Aspergillus awamori, Aspergillus foetidus, Aspergillus japonicus, Aspergillus nidulans, Aspergillus niger or Aspergillus oryzae cell. In another most preferred embodiment, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In an even most preferred embodiment, the filamentous fungal parent cell is a Fusarium venenatum (Nirenberg sp. nov.) cell. In another most preferred embodiment, the filamentous fungal host cell is a Humicola insolens, Humicola lanughosa, Mucor miehei, Myceliophthora thermophila, Neurospora crassa, Penicillium purpurogenum, Thielavia terrestris, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Methods of Production

The present invention also relates to methods for producing a polypeptide of the present invention comprising (a) cultivating a strain, which in its wild-type form is capable of producing the polypeptide; and (b) recovering the polypeptide. Preferably, the strain is of the genus Aspergillus, and more preferably Aspergillus orzae and Aspergillus niger.

The present invention also relates to methods for producing a polypeptide of the present invention comprising (a) cultivating a host cell under conditions conducive for production of the polypeptide; and (b) recovering the polypeptide.

In the production methods of the present invention, the cells are cultivated in a nutrient medium suitable for production of the polypeptide using methods known. in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J. -C. Janson and Lars Ryden, editors, VCH Publishers, New York, 1989).

Applications

Therapeutic formulations of the antibodies produced in accordance with the present invention may be formulated as known in the art.

The formulation may contain more than one active compound as necessary for the particular indication being treated. For example, it may be desirable to provide another type of antibody, and/or the composition may comprise a cytotoxic agent, a cytokine or a growth inhibitory agent. is The formulations to be used for in vtivo administration must be sterile. This is readily accomplished by e.g. filtration through sterile filtration membranes.

Another application of the antibodies is chimeric proteins consisting of the binding part of antibodies and enzymes. In this way catalytic biomolecules can be designed that have two binding properties, one of the enzyme and the other of the antibody. This may result in enzymes that have superior activity.

### EXAMPLES

### Example 1

Construction of the Aspergillus strain Jal355:

BECh2 is described in WO 00/39322 which further refer to patent WO 98/12300 (describes JaL228).
pJaL173 is described in WO 98/12300
pJaL335 is described in WO 98/12300

For removing the defect pyrG gene resident in the alkaline protease gene in the A. oryzae strain BECh2 the following was done:

Isolation of a pyrG⁻ A. oryzae strain, ToC1418:

The A. oryzae strain BECh2 was screened for resistance to 5-flouro-orotic acid (FOA) to identify spontaneous pyrG mutants. One strain, ToC1418, was identified as being pyrG⁻. ToC1418 is uridine dependent, therefore it can be transformed with the wild type pyrG gene and transformants selected by the ability to grow in the absence of uridine.

Construction of a pyrG Plus A. oryzae strain, JaL352:

The mutation in the defect pyrG gene resident in the alkaline protease gene was determined by sequencing. Chromosomal DNA from A. oryzae strain BECh2 was prepared by PCR using primers 104025 and 104026.

104025 (SEQ ID NO. 2): 5′-CCTGAATTCACGCGCGCCAACATGTCTTCCAAGTC,
and
104026 (SEQ ID NO. 3): 5′-GTTCTCGAGCTACTTATTGCGCACCAACACG

A 933 bp fragment was amplified containing the coding region of the defect pyrG gene. The 933 bp fragment was purified and sequenced with the following primers:

Primer 104025, primer 104026, primer 104027
(Seq ID No. 4): 5′-ACCATGGCGGCACTCTGC,
primer 104028 (Seq ID No. 5):
5′-GAGCCGTAGGGGAAGTCC,
primer 108089 (Seq ID No. 6):
5′-CTTCAGACTGAACCTCGCC,
and
primer 108091 (Seq ID No. 7):
5′-GACTCGGTCCGTACATTGCC.

Sequencing shows that an extra base, a G, was inserted at position 514 in the pyrG-coding region (counting from the A in the start codon of the pyrG gene), thereby creating a frame-shift mutation.

To make a wild type pyrG⁻ gene out of the defect pyrG gene resident in the alkaline protease the A. oryzae pyrG⁻ strain ToC1418 was transformed with 150 pmol of the oligo-nucleotide 5′- CCTACGGCTCCGAGAGAGGCCTTTTGATCCTTGCGGAG-3′ (SEQ ID NO. 8), using standard produres. The oligo-nucleotide may advantageously be phosphorylated at the 5′-end. The oligo-nucleotide restores the pyrG reading frame, but at the same time a silence mutation is introduced thereby creating a Stul restriction endonuclease site. Transformants were then selected by their ability to grow in the absence of uridine. After re-isolation chromosomal DNA was prepared from 8 transformants. To confirm the changes a 785 bp fragment was amplified by PCR using the primers 135944 (Seq ID No. 9): 5′-GAGTTAGTAGTTGGACATCC and primer 108089, which is covering the region of interest. The 785 bp fragment was purified and sequenced with the primers 108089 and 135944. One strain having the expected changes was named JaL352.

Isolation of a pyrG⁻ A. oryzae strain, JaL355:

For removing the pyrG gene resident in the alkaline protease gene JaL352 was transformed by standard procedure with the 5.6 kb BamHI fragment of pJaL 173 harbouring the 5′ and 3′ flanking sequence of the A. oryzae alkaline protease gene. Protoplasts were regenerated on non-selective plates and spores were collected. About 10⁹ spores were screened for resistance to FOA to identify pyrG mutants. After re-isolation chromosomal DNA was prepared from 14 FOA resistance transformants. The chromosomal DNA was digested with Bal I and analysed by Southern blotting, using the 1 kb ³²P-labelled DNA Bal I fragment from pJaL173 containing part of the 5′ and 3′ flanks of the A. oryzae alkaline protease gene as the probe. Strains of interest were identified by the disappearance of a 4.8 kb Bal I band and the appearance of a 1 kb Bal I band. Probing the same filter with the 3.5 kb ³²P-labelled DNA Hind III fragment from pJaL335 containing the A. oryzae pyrG gene results in the disappearance of the 4.8 kb Bal I band in the strains of interest. One strain resulting from these transformants was named JaL355.

### Example 2

Construction of Plasmids used for Expression:

In order to improve expression of a gene of interest on an expression plasmid, it may be desirable to reduce the expression of the gene marker used for selection, exemplified here by the pyrG gene. By cultivating a host cell harbouring an expression plasmid comprising a selection gene, that has reduced expression, under normal selective pressure results in a selection for a host cell which has an increased plasmid copy number, thus achieving the total expression level of the selection gene necessary for survival. The higher plasmid copy-number, however, also results in an increased expression of the gene of interest.

One way of decreasing the expression level of the selection gene is to lower the mRNA-level by either using a poorly transcribed promoter or decreasing the functional half-life of the mRNA. Another way is to reduce translation efficiency of the mRNA. One way to do this is to mutate the Kozak-region (Kozak M Gene, Vol. 234 (2) pp. 187-208 (1999)). This is a region just upstream of the initiation codon (ATG), which is important for the initiation of translation.

Plasmid pENI2155 comprises a bad kozak region upstream of the pyrG gene, and is constructed as follows:

Using plasmid pENI1861 (the construction of which is described below) as template, and PWO polymerase (conditions as recommended by manufacturer); two PCR-reactions were made using primer 141200j1 and 270999J9 in the one PCR-reaction and primers 141200J2 and 290999J8 in another PCR-reaction:

141200J1 (SEQ ID NO:10):
5′ ATCGGTTTTATGTCTTCCAAGTCGCAATTG
141200J2 (SEQ ID NO:11):
5′ CTTGGAAGACATAAAACCGATGGAGGGGTAGCG
270999J8 (SEQ ID NO:12):
5′ TCTGTGAGGCCTATGGATCTCAGAAC
270999J9 (SEQ ID NO:13):
5′ GATGCTGCATGCACAACTGCACCTCAG

The PCR fragments were purified from a 1% agarose gel using QIAGEN™ spin columns. A second PCR-reaction was run using the two fragments as template along with the primers 270999J8 and 270999J9. The PCR-fragment from this reaction was purified from a 1% agarose gel as described; the fragment and the vector pENI1849 (containing a lipase gene as expression reporter) were cut with the restriction enzymes Stul and Sphl, the resulting fragments were purified from a 1% agarose gel using conventional methods.

The purified fragments were ligated and transformed into the E. coli strain DH10B. Plasmid DNA from one of the transformants was isolated and sequenced to confirm the introduction of a mutated Kozak region: GGTTTTATG (rather than the wildtype: GCCAACATG). This Plasmid was denoted: pENI2155.

Aspergillus cells were transformed with plasmid pENI1849 (control wildtype plasmid), and pENI2155 (mutated Kozak region upstream of the pyrG gene). Approximately 1 microgram of pENI1849 and pENI2155 were transformed into A. oryzae Jal355 (JaL355 is a derivative of A. oryzae A1560 wherein the pyrG gene has been inactivated, as described in WO 98/01470; transformation protocol as described in WO 00/24883). The transformants were incubated for 4 days at 37° C.

24 transformants from the pENI2155 transformation and 12 transformants from pENI1849 were inoculated in a 96 well microtiter plate containing 1×Vogel medium and 2% maltose (Methods in Enzymology, vol. 17, p. 84). After 4 days growth at 34° C., the culture broth was assayed for lipase activity using pnp-valerate as a lipase substrate.

A 10 microliter aliquot of media from each well was added to a microtiter well containing 200 microliter of a lipase substrate of 0.018% p-nitrophenylvalerate, 0.1% Triton X™-100, 10 mM CaCl₂, 50 mM Tris pH 7.5. Lipase activity was assayed spectrophotometrically at 15-seconds intervals over a five minute period, using a kinetic microplate reader (Molecular Device Corp., Sunnyvale Calif.), using a standard enzymology protocol (e.g., Enzyme Kinetics, Paul C. Engel, ed., 1981, Chapman and Hall Ltd.). Briefly, product formation is measured during the initial rate of substrate turnover and is defined as the slope of the curve calculated from the absorbance at 405 nm every 15 seconds for 5 minutes. The arbitrary lipase activity units were normalized against the transformant showing the highest lipase activity. For each group of thirty transformants an average value and the standard deviations were calculated. Given in arbitrary units the average lipase activity and relative standard deviation was:

1849 Transformant: 65±14

2155 Transformant: 120±22

Clearly there is nearly a doubling of lipase expression in the 2155 transformant, wherein the mutated Kozak region was introduced in front of the selection gene pyrG.

Plasmid pENI1861 was made in order to have the state of the art Aspergillus promoter in the expression plasmid, as well as a number of unique restriction sites for cloning. A PCR fragment (Approx. 620 bp) was made using plasmid pMT2188 (the construction of pMT2188 is described below) as template and the following primers:

051199J1 (SEQ ID NO:14): 5- CCTCTAGATCTCGAGCTCGGTCACCGGTGGCCTCCG
CGGCCGCTGGATCCCCAGTTGTG
1298TAKA (SEQ ID NO:15): 5′-GCAAGCGCGCGCAATACATGGTGTTTTGATCAT

The fragment was cut with BssHII and Bg/II, and cloned into pENI1849 which was also cut with BssHII and Bgl II. The cloning was verified by sequencing.

Plasmid pENI1849 was made in order to truncate the pyrG gene to the essential sequences for pyrG expression, in order to decrease the size of the plasmid, thus improving transformation frequency. A PCR fragment (Approx. 1800 bp) was made using pENI1299 (described in WO 00/24883 FIG. 2 and Example 1) as template and the following primers: 270999J8 (SEQ ID NO:12), and 270999J9 (SEQ ID NO:13)

The PCR-fragment was cut with the restriction enzymes Stul and Sphl, and cloned into pENI1298 (described in WO 00/24883 FIG. 1 and Example 1), also cut with Stul and Sphl; the cloning was verified by sequencing.

Plasmid pMT2188 was based on the Aspergillus expression plasmid pCaHj 483 (described in WO 98/00529), which consists of an expression cassette based on the Aspergillus niger neutral amylase II promoter fused to the Aspergillus nidulans-triose. phosphate isomerase non translated leader sequence (Pna2/tpi) and the A. niger amyloglycosidase terminater (Tamg). Also present on the pCaHj483 is the Aspergillus selective marker amdS from A. nidulans enabling growth on acetamide as sole nitrogen source. These elements are cloned into the E. coli vector pUC19 (New England Biolabs). The ampicillin resistance marker enabling selection in E. coli of pUC19 was replaced with the URA3 marker of Saccharomyces cerevisiae that can complement a pyrF mutation in E. coli, the replacement was done in the following way:

The pUC19 origin of replication was PCR amplified from pCaHj483 with the primers:

142779 (SEQ ID NO:16):
5′-TTGAATTGAAAATAGATTGATTTAAAACTTC
142780 (SEQ ID NO:17):
5′-TTGCATGCGTAATCATGGTCATAGC

Primer 142780 introduces a Bbul site in the PCR fragment. The Expand™ PCR system (Roche Molecular Biochemicals, Basel, Switserland) was used for the amplification following the manufacturers instructions for this and the subsequent PCR amplifications.

The URA3 gene was amplified from the general S. cerevisiae cloning vector pYES2 (Invitrogen corporation, Carlsbad, Calif., USA) using the primers:

140288 (SEQ ID NO:18):
5′-TTGAATTCATGGGTAATAACTGATAT
142778 (SEQ ID NO:19):
5′-AAATCAATCTATTTTCAATTCAATTCATCATT

Primer 140288 introduces an EcoRl site in the PCR fragment. The two PCR fragments were fused by mixing them and amplifying using the primers 142780 and 140288 in the splicing by overlap method (Horton et al (1989) Gene, 77, 61-68).

The resulting fragment was digested with EcoRl and Bbul and ligated to the largest fragment of pCaHj 483 digested with the same enzymes. The ligation mixture was used to transform the pyrF⁻ E. coli strain DB6507 (ATCC 35673) made competent by the method of Mandel and Higa (Mandel, M. and A. Higa (1970) J. Mol. Biol. 45, 154). Transformants were selected on solid M9 medium (Sambrook et. al (1989) Molecular cloning, a laboratory manual, 2. edition, Cold Spring Harbor Laboratory Press) supplemented with 1 g/l casamino acids, 500 microgram/l thiamine and 10 mg/l kanamycin.

A plasmid from a selected transformant was termed pCaHj527. The Pna2/tpi promoter present on pCaHj527 was subjected to site directed mutagenesis by a simple PCR approach. Nucleotide 134-144 was altered from GTACTAAAACC to CCGTTAAATTT using the mutagenic primer 141223. Nlucleotide 423-436 was altered from ATGCAATTTAAACT to CGGCAATTTAACGG using the mutagenic primer 141222. The resulting plasmid was termed pMT2188.

141223 (SEQ ID NO:20): 5′-GGATGCTGTTGACTCCGGAAATTTAACGGTTTGGTCTTGCATCCC
141222 (SEQ ID NO:21): 5′-GGTATTGTCCTGCAGACGGCAATTTAACGGCTTCTGCGA
ATCGC

Decreasing the Activity and Stability of the OMP Decarboxylase

In order to improve expression of a gene of interest from a plasmid, it may be desirable to reduce the stability and/or the activity of the protein encoded by the selection gene (for instance the pyrG gene) as already mentioned in Example 1.

One way of decreasing the stability of the protein encoded by the selection gene is to add a “degron” motif to the protein (Dohmen R. J., Wu P., Varshavsky A., (1994) Science vol 263 p.1273-1276). Another way is to identify structurally important conserved amino acid residues, based on alignment to homologous proteins or based on a model-structure of the protein (if available). These amino acids may then be mutated to decrease the stability and/or the activity of the enzyme.

A protein alignment was made with the protein sequence: swissprot_dcop_aspng (the OMP decarboxylase encoded by the pyrG gene on plasmid pENI2155) to the following database entries: Swissprot_dcop-aspor, geneseqp_r05224, geneseqp_y99702, tremblnew_aag34761, swissprot_dcop_phybl, remtermbl_aab01165, remtembl_aab 16845, and sptrembl_q9uvz5.

The alignment was done using the program ClustalW (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680).

Based on these alignments and the structure of the related Bacillus subtilis OMP decarboxylase (Appleby t., Kinsland C., Begley T. P., Ealick S. E. (2000), Proc. Natl. Acad. Sci. USA, vol 97 p. 2005-2010) the following conserved residues were identified as potentially structurally important, and as such suitable targets for mutation: P50, F91, F96, N101, T102, G128, G222, D223, G239. A number of mutagenic primers were constructed, and were phosphorylated using T4 polynucleotide kinase (New England Biolabs).

P50-260301j1: (SEQ ID NO:22)
5′-ACAGGACTCGGTNCGTACATTGCCGTG
F91-260301j2: (SEQ ID NO:23)
5′-AATTTCCTCATCTNCGAAGATCGCAAG
F96-260301j3: (SEQ ID NO:24)
5′-GAAGATCGCAAGTNCATCGATATCGGA
N101,T102-260301j4: (SEQ ID NO:25)
5′-ATCGATATCGGANACANCGTCCAAAAG
CAG
G128-260301j5: (SEQ ID NO:26)
5′-AGTATTCTGCCCGNTGAGGGTATCGTC
G222, D223-260301j6: (SEQ ID NO:27)
5′-CTCTCCTCGAAGGNTNACAAGCTGGG
ACAG
G239-230301j7: (SEQ ID NO:28)
5′-GCTGTTGGACGCGNTGCCGACTTTATT

Seven individual PCR/ligation reactions were performed (as described by Sawano A., Miyawaki A. (2000) Nucleic Acid Research vol. 28 e78) using pENI2155 as template, and 1 microliter DNA from each of the seven libraries was transformed into the E. coli strain DH10B. Approximately 1000 E. coli clones were obtained from each library. DNA preparation was made from each library and the DNA was pooled together (named pBIB16).

The Aspergillus strain MT2425 (a pyrG minus strain, which gives small transformant-clones, when grown on the selection plates) was transformed with 1 microgram of the pBIB16 DNA and 10 microgram herring sperm DNA (carrier DNA) pr. 100 microliter protoplast using standard procedures.

The transformed protoplast were spread on selection plates (2% maltose (inducing small morphology and lipase expression), 10 mM NaNO₃, 1.2 M sorbitol, 2% bacto agar, and standard salt solution).

After 5 days of growth, an overlay (containing 0.004% brilliant green, 2.5% olive oil, 1% agar, 50 mM TRIS pH 7.5 treated with a mixer for 1 min. (Ultrathorax™ Type T25B, IKA Labortechnic, Germany)) was poured onto the Aspergillus transformant clones. The plates where incubated over night at room temperature.

Twenty of the clones having the highest activity towards olive oil were inoculated in to 200 microliter YPM in a 96 well microtiter plate. After 4 days of growth at 34° C., the culture broths were assayed for lipase activity using pnp-valerate as described above.

The 6 transformants giving the highest activity in the lipase assay were inoculated in 5 ml YPM. DNA was isolated and transformed into the E. coli strain DH10B, thus rescuing the plasmid (as also described in WO 00/24883). Two pyrG variants were identified:
1) F96S; the plasmid was denoted pENI2343, and
2) T102N; the plasmid was denoted pENI2344.

Approx. 2 microgram of each of the plasmids pENI2155, pENI2343 and pENI2344 were transformed into an Aspergillus oryzae pyrG-minus mutant denoted Jal355, and an Aspergillus niger pyrG-minus mutant denoted Mbin 115, using standard procedures.

The transformed protoplasts were spread on selection plates (2% maltose 10 mM NaNO₃, 1.2 M sorbitol, 2% bacto agar, salt solution). After 4 days of growth, very poor sporulation was seen for the pEN 12343 Jal355 transformants, and no transformants were seen for MBIN115 transformed with pENI2343.

6 independent transformants of each plasmid transformation were inoculated into 200 microliter 1×Vogel, 2% maltose in a 96-well microtiter plate. After 4 days growth at 34° C., the culture broths were assayed for lipase activity. The results are given in the table below as relative lipase units with relative standard deviation, and are averages of the activity of the independent clones.

| | Jal355 | Mbin115 |
|---|---|---|
| pENI2155 (wt) | 48 ± 8% | 7 ± 14% |
| pENI2343 (F96S) | 49 ± 15% | No growth |
| pENI2344 (T102N) | 71 ± 13% | 80 ± 11% |

The expression of lipase from the pENI2343 transformants was very high compared to the fungal biomass in the wells, which was very poor (less than 1/10 of the other transformants). An approx. 1.5-fold increase in lipase expression level is seen for the Jal355 transformants, and an approx. 11-fold increase is seen in the Mbin115 transformants, when comparing the pENI2155 transformants with the pENI2344 transformants.

Thus the pyrG T102N mutation leads to an increase in lipase expression, likely due to an increased plasmid copy number, which is selected for because of the unstable, less active OMP decarboxylase encoded by the selection gene pyrG.

In order to evaluate plasmid stability, a screen was set up to evaluate the percentage of spores containing a stably episomaly replicated plasmid (comprising a pyrG selection gene).

Two DNA libraries were constructed. The first library was cloned into a plasmid comprising the wildtype pyrG gene as selection gene, whereas the second library was cloned into a plasmid comprising a mutated pyrG gene which comprised a mutated Kozak region and a T102N mutation.

A spore suspension was made from each library and plated on plates (2% maltose 10 mM NaNO₃, 1.2 M sorbitol, 2% bacto agar, salts, with or without 20 mM uridine). The plates were grown for 3 days at 37° C. Results are shown in the table below.

| Selection gene | − uridine | + uridine | % viable spores |
|---|---|---|---|
| Wildtype pyrG | 11 | 83 | 13 |
| Mutant (Kozak/T102N) pyrG | 36 | 63 | 57 |

Evidently a much larger fraction of the spores contain a plasmid, when using the mutated (Kozak/T102N) pyrG gene.

Construction of PENI2151:

pENI1902 and pENI1861 were cut with HindIII, and pENI1902 was treated with alkaline phosphatase.

A fragment of 2408 bp from pENI1861 was purified from a 1% gel and ligated to the vector of pENI1902 purified from a 1% gel thus creating pENI2151.

Construction of PENI2207 (Having a Poor Kozak-Region Upstream of pyrG):

pENI2151 and pENI2155 were cut with Stul and Sphl.

A fragment of 2004 bp from pENI2155 was purified from a 1% gel and ligated with the cut pENI2151 , also purified from a 1% gel, thus creating pENI2207.

Construction of PENI2229 (Having Additional Restriction Sites in Linker):

A PCR was run using oligo 2120201J1 and 1298-TAKA along with pENI2151 as template.

The PCR fragment (650 bp) as well as pENI2207 were cut BssHII and BgIII. The vector and the PCR fragment were purified from a 1% gel and ligated thus creating pENI2229.

1298-TAKA (SEQ ID NO.15): 5′-GCAAGCGCGCGCAATACATG
GTGTTTTGATCAT
210201J1 (SEQ ID NO. 29): 5′-GCCTCTAGATCTCCCGGGCG
CGCCGGCACATGTACCAGGTCTTAAGCTCGAGCTCGGTCACCGGTGGCC

Construction of pENI2376 Having a Poor Kozak and Impaired pyrG Gene:

The plasmid pENI 2344 was cut Sphl and Stul and the DNA fragment (2004 bp) containing the pyrG gene was isolated from a 1% agarose gel.

The plasmid pENI 2229 was cut Sphl and Stul and the Vector fragment was isolated from a 1% agarose gel.

The vector fragment from pENI2229 and the pyrG containing fragment from pENI2344 was ligated, thus creating pENI2376.

Construction of pENI2516:

The plasmid pENI2376 was cut HindIII and the major vector fragment of 6472 bp was ligated, thus creating pENI2516.

### Example 3

Herceptin is a human antibody, which is used for curing breast cancer. This is a very expensive product, and it would be cheaper to produce a similar product in filamentous fungi having a very high expression potential.

Based on the amino acid sequence of the human heavy chain fragment of Herceptin, a gene was constructed, which has the same codon usage as is found for highly expressed genes in Aspergillus.

Primers as shown in FIG. 1 were designed from the above DNA sequence in a way so that the gene encoding the heavy chain variable domain of Herceptin could be synthesized. The relative positions of the primers are shown in FIG. 1.

Construction of pENI2716:

The primers 230402j3 (10 pmol), 230402j4 (2 pmol), 230402j7 (10 pmol) and 230402j8 (2 pmol) were mixed in a total of 20 μl and a PCR reaction (94° C. 5 min, 25 cycles of (94° C. 30 sec, 50° C. 30 sec, 72° C. 1 min) 72° C. 2 min) was run using TGO-polymerase and buffer (Roche).

230402j3 (SEQ ID NO 30): 5′-ACCTTCACCGACTACACGATGG
ACTGGGTCCGGCAGGCGCCGGGCAAGGGCCTGGAGTG
230402j4 (SEQ ID NO 31): 5′-CCGGGCAAGGGCCTGGAGTGGG
TCGCGGACGTGAACCCGAACTCCGGCGGGTCGATCTACAACCAGCGCT
230402j7 (SEQ ID NO 32): 5′-AGACGGCGGTGTCCTCCGCCCG
GAGGGAGTTCATCTGCAGGTACAGCGTGTTCTTCGACC
230402j8 (SEQ ID NO 33): 5′-GTACAGCGTGTTCTTCGACCGG
TCGACCGAGAGCGTGAACCGGCCCTTGAAGCGCTGGTTGTAGATCGAC

The generated PCR fragment (see FIG. 1) was cloned into pCR4TOPO blunt vector (Invitrogen, as recommended by manufacture), and transformed into TOP10 E. coli cells. DNA-prep was made from E. coli transformants, and sequenced. The plasmid with the correct sequence, encoding a fragment of the heavy chain variable domain of herceptin, was named pENI2716.

Construction of pENI2769:

The primers 230402J1 (10 pmol), 230402j2 (2 pmol), 230402j5 (10 pmol) and 230402j6 (2 pmol) and the plasmid pENI2716 were mixed in a total of 20 μl and a PCR reaction (94° C. 5 min, 25 cycles of (94° C. 30 sec, 50° C. 30 sec, 72° C. 1 min) 72° C. 2 min) was run using TGO-polymerase and buffer (Roche).

230402J1 (SEQ ID NO 34): 5′-GAGGTCCAGCTCGTCGAGTCCG
GCGGCGGCCTCGTGCAGCCGGGGGGCTCGCTGCGGCTC
230402j2 (SEQ ID NO 35): 5′-CGGGGGGCTCGCTGCGGCTCTC
CTGCGCCGCGTCGGGCTTCACCTTCACCGACTACACGA
230402j5 (SEQ ID NO 36): 5′-ATCGAGCCGCGGCTACGAGGAG
ACGGTGACCAGGGTGCCCTGGCCCCAGTAGTCGAAGTAGAACGACGGGCC
230402j6 (SEQ ID NO 37): 5′-TCGAAGTAGAACGACGGGCCGA
GGTTCCGGGCGCAGTAGTAGACGGCGGTGTCCTCCGCC

The generated PCR fragment (see FIG. 1) was cloned into pCR4TOPOblunt vector (Invitrogen, as recommended by manufacture), and transformed into TOP10 coli cells. DNA-prep was made from E. coli transformants, and sequenced. The plasmid with the correct sequence, encoding the full heavy chain variable domain of herceptin, was named pENI2769.

### Example 4

### Construction of the Expression Plasmids pENI-Herceptin1 and pENI-Herceptin2 for the Expression of the Heavy Chain Variable Domain of Herceptin

Using primer and 230402j1 and 230402j5 along with a template (pENI2769) a PCR reaction (94° C. 5 min, 25 cycles of (94° C. 30 sec, 50° C. 30 sec, 72° C. 1 min) 72° C. 2 min) was run using TGO-polymerase and buffer (Roche). The resulting PCR fragment encodes the heavy chain variable domain of herceptin.

PCR of the Meripilus gigantes Cellulose Binding Domain

Using primer 090103j1 and 230402J9 along with the plasmid isolated from a strain deposited at DSM (DSM9971) a PCR reaction (94° C. 5 min, 25 cycles of (94° C. 30 sec, 50° C. 30 sec, 72° C. 1 min) 72° C. 2 min) was run using TGO-polymerase and buffer (Roche). DSM 9971 is a yeast Saccharomyces cerevisiae, comprising an endoglucanase cloned in the expression plasmid pYES 2.0 (Invitrogen). Also comprised in said plasmid is the Meripilus giganteus cellulose binding domain. Said yeast has been deposited according to the Budapest Treaty on the International Recognition of the Deposit of Microorganisms for the Purposes of Patent Procedure at the Deutshe Sammlung von Mikroorganismen und Zellkulturen GmbH., Mascheroder Weg 1b, D-38124 Braunschweig Federal Republic of Germany, (DSMA).
Deposit date: 11.05.95
Depositor's ref.: NN49008
DSM designation: Saccharomyces cerevisiae DSM No. 9971

The resulting PCR fragment contains the TAKA-promoter, and the Meripilus giganteus cellulose binding domain, which is well expressed in Aspergillus.

230402J9 (SEQ ID NO 38): 5′-GACTCGACGAGCTGGACCTCCG
AGCCAGGGCACGCGGACGG
090103j1 (SEQ ID NO 39): 5′-GTAGACGGATCCACCATGAAGG
CGATCCTCTCTCTCGC

The PCR fragments generated with 090103j1/230402j9 and 230402j1/230402j5 were mixed and a new PCR reaction (94° C. 5 min, 25 cycles of (94° C. 30 sec, 50° C. 30 sec, 72° C. 1 min) 72° C. 2 min) was run using TGO-polymerase and buffer (Roche) with primer 090103j1 and 230402j5. The resulting PCR fragment encodes the well expressed Meripilus giganteus cellulose binding domain fused to the heavy chain variable domain of herceptin, in order to ensure fine expression of the heavy chain variable domain of herceptin.

The resulting PCR fragment was cut with BamHI and SacII, and cloned into the expression vector pENI2376 cut with BamHI and SacII for expression in Aspergillus libraries, thus creating pENI-Herceptin1.

The resulting PCR fragment was cut with BamHI and SacII, and cloned into the expression vector pENI2516 cut with BamHI and SacII for expression in aspergillus, thus creating pENI-herceptin2.

pENI-herceptin2 was transformed into the Aspergillus strain JaL355 as mentioned in example 2. Twenty Aspergillus transformants were inoculated in to 200 microliter YPM in a 96 well microtiter plate. After 4 days of growth at 34° C., 20 microliter of the culture broths was run on a 16% SDS-PAGE. s Transformants expressing the heavy chain variable domain of herceptin was identified as bands on a 16% SDS-page.

### Example 5

### Fermentation of Aspergillus transformant expressing the Heavy Chain Variable Domain of Herceptin from pENI-herceptin2

The Aspergillus transformant with the best expression of Herceptin was inoculated in a shake-flask containing 100 ml G2-gly (Yeast Extract 18 g/L, Glycerol 87% 24 g/L, Pluronic PE-6100 0.1 ml/L) and grown over night at 30° C. on shaking at 275 rpm. Next day 2 ml of culture is used to inoculate a shake-flask containing 100 ml MDU-2B (Maltose 45 g/L, Magnesium-sulfat 1 g/L, Sodium chlorid 1 g/L, Potasium sulfat 2 g/L, Yeast Extract 7 g/L, trace metal (KU6) 0.5 ml/L, Pluronic PE 6100 0.1 ml/L)+1% urea. 10 flask were inoculated and grown for 72 hours at 30° C. on shaking at 275 rpm. Trace metal: ZnCI₂ 6,8 g/L, CuSO₄.5H₂O 2,5 g/L, NiCl₂.6H₂O 0,24 g/L, FeSO₄.7H₂O 13,9 g/L, MnSO₄.H₂O 8,45 g/L, citrate C₆H₈O₇.H₂O 3 g/L.

### Example 6

### Construction of the Expression Vector PENI-herceptin3 with a Sequence Encoding the Thermomyces lanuginosa lipase Signal Peptide Upstream of Heavy Chain Variable Domain of Herceptin, and Transformation into A. orvzae

Using primer 081102J5 and 211102j1 along with a template (pENI2769) a PCR reaction (94° C. 5 min, 25 cycles of (94° C. 30 sec, 50° C. 30 sec, 72° C. 1 min) 72° C. 2 min) was run using TGO-polymerase and buffer (Roche). The resulting PCR fragment encodes the heavy chain variable domain of herceptin.

081102J5 (SEQ ID NO 40): 5′-GCCTTGGCTAGCCCTATTCGTC
GAGAGGTCCAGCTCGTCGAGTCC
211102j1 (SEQ ID NO 41): 5′-CACGAGCTCGAGCCGCGGCTAC
GAGGA

The generated PCR fragment and the plasmid pENI1163 (WO 99/42566) was cut Nhel and Xhol. The PGR fragment and the vector plasmid (pENI1163) was purified from 1.5% agarose gel, ligated o/n and transformed into the Coli strain DH10B. The resulting plasmid, pENI-herceptin3, was transformed into the Aspergillus strain Bech2 (see above), and screened for expression of heavy chain variable domain of herceptin, as described above.

### Example 7

### Construction of the Expression Vector pENI-herceptin4 with a Sequence Encoding the Thermomyces lanuginosa Lipase Signal Peptide Upstream of Heavy Chain Variable Domain of Herceptin and a Lipase-Encoding Gene Downstream

Using primer 081102J5 and 030103j1 along with a template (pENI2769) a PCR reaction (94° C. 5 min, 25 cycles of (94° C. 30 sec. 50° C. 30 sec, 72° C. 1 min) 72° C. 2 min) was run using TGO-polymerase and buffer (Roche). The resulting PCR fragment encodes the heavy chain variable domain of herceptin.

081102J5 (SEQ ID NO 42): 5′-GCCTTGGCTAGCCCTATTCGTC
GAGAGGTCCAGCTCGTCGAGTCC
030103j1 (SEQ ID NO 43): 5′-GTCAGCGCTAGCCGAGGAGACG
GTGACCAGGGTGCC

The generated PCR fragment and the plasmid pENI1163 (WO 99/42566) was cut Nhel. The PCR fragment and the vector plasmid (pENI1163) was purified from 1.5% agarose gel, ligated over night and-transformed into the coli strain DH10B. The resulting plasmid (pENI-herceptin4) was sequenced and transformed into the Aspergillus strain Bech2 (see above), and screened for expression of heavy chain variable domain of herceptin, by assaying for lipase activity (see patent WO 00/24883 A1).

### Example 8

### Construction of PENI-herceptin5 for Library Screening in Aspergillus

Using primer 1298-taka (see above) and 991213j5 along with a template (pENI-herceptin4) a PCR reaction (94° C. 5 min, 25 cycles of (94° C. 30 sec, 50° C. 30 sec, 72° C. 1 min) 72° C. 2 min) was run using TGO-polymerase and buffer (Roche). The resulting PCR fragment encodes the heavy chain variable domain of herceptin cloned upstream in a translational fusion with a lipase gene.

991213j5 (SEQ ID NO 44): 5′-CCTCTSGATCTCGAGCTCGGTC
ACCGGTGGCCTCCGCGGCCGCTGCGCCAGGTGTCAGTCACCCTC

The generated PCR fragment and the plasmid pENI2376 (WO 99/42566) was cut BamHI and SacII. The PCR fragment and the vector plasmid (pENI2376) was purified from 1.5% agarose gel, ligated o/n and transformed into the Coli strain DH10B. The resulting plasmid (pENI-herceptin5) was sequenced and transformed into the Aspergillus strain jal355 (see above), and screened for expression of heavy chain variable domain of herceptin, by assaying for lipase activity (see patent WO 00/24883 A1).

### Example 9

### Screening for Increased Solubility and Production of Heavy Chain Variable Domain Expressed from PENI-herceptin5

In order to improve expression and solubility heavy chain variable domain, it is obvious to mutate amino acid residues involved in the contact between the heavy chain and the light chain. Potentially any amino acid change could do so, by changing the overall protein structure slightly. The amino acids residues should preferably be mutated to hydrophilic residues, such as K, R, H, D, E, G, N, Q, C, S, T or Y. The positions to be mutated should in the given example preferably be: V37, Q39, G44, L45, W47, Y95 and W109.

In order to increase expression and solubility the following screen was performed The following phosphorylated primers were designed, in which X designates naturally occurring amino acids and the amino acid positions refer to SEQ ID NO 1.

301202j1 V37X, Q39X: (SEQ ID NO 45)
5′-ACGATGGACTGGNNSCGGNNSGCGCCGGGCAAG
301202j2 G44X, L45X, W47X: (SEQ ID NO 46)
5′-GCGCCGGGCAAGNNSNNSGAGNNSGTCGCGGACGTG
301202j3 Y95 X: (SEQ ID NO 47)
5′-ACCGCGGTCTACNNSTGCGCCCGGAAC
301202j4 W109X: (SEQ ID NO 48)
5′-ACTTCGACTACNNSGGCCAGGGCACC
7887: (SEQ ID NO 49)
5′-GAA TGA CTT GGT TGA GTA CTC ACC AGT CAC

(Thus changing the MIul site found in the ampicillin resistance gene and used for cutting to a Scal site).

A library was made in E. coli using the plasmid pENI-herceptin5 as template, the mutation oligoes 301202j1, 301202j2, 301202j3, 301202j4 and oligo7887 as selection oligo along with the the commercial kit, Chameleon double-stranded, site-directed mutagenesis kit can be used according to the manufacturer's instructions (Stratagene).

The resulting E. coli library was transformed in the Aspergillus strain Jal355 (as mentioned in patent WO 00/24883 A1).

JaL355 was transformed with library using standard procedures, cf., as described in WO 98/01470. The cells were then cultured on Cove plates at 37° C.

Transformants appeared after three days incubation at a transformation frequency of 10⁴-10⁵/μg DNA

5000 independent transformants were inoculated into a 384-well microtiter dish containing 40 μl minimal media of 1×Vogel, 2% maltose (e.g., Methods in Enzymology, Vol. 17 p. 84) in each well.

After three days of incubation at 34° C., media from the cultures in the microtiter dish were assayed for lipase activity. A 5 μl aliquot of media from each well was added to a microtiter well containing 40 μl of a lipase substrate of 0.018% p-nitrophenylbutyrate, 0.1% Triton X-100, 10 mM CaCl₂, 50 mM Tris pH 7.5. Activity was assayed spectrophotometrically at 15-second intervals over a five minute period, using a kinetic microplate reader (Victor 2, Wallac), using a standard enzymology protocol (e.g., Enzyme Kinetics, Paul C. Engel, ed., 1981, Chapman and Hall Ltd.) Briefly, product formation is measured during the initial rate of substrate turnover and is defined as the slope of the curve calculated from the absorbance at 405 nm every 15 seconds for 5 minutes. The 50 strains expressing the highest level of lipase were isolated. The increased lipase expression was taken as an indication of increased expression and solubility of the heavy chain variable domain. Media from these 50 strains were further analysed by SDS-PAGE to identify the best expression.

### Example 10

Screening for Increased Solubility and Production of the Heavy Chain Variable Domain Expressed from pENI-herceptin1.

In order to improve expression and solubility heavy chain variable domain, it is obvious to mutate amino acid residues involved in the contact between the heavy chain and the light chain. Potentially any amino acid change could do so, by changing the overall protein structure slightly. The amino acids residues should preferably be mutated to hydrophilic residues, such as K, R, H, D, E, G, N, Q, C, S, T or Y. The positions to be mutated should in the given example preferably be: V37, Q39, G44, L45, W47, Y95 and W109.

In order to increase expression and solubility the following screen was performed The following phosphorylated primers were designed (same as in example 9 above):

301202j1 V37X, Q39X: (SEQ ID NO. 45)
5′-ACGATGGACTGGNNSCGGNNSGCGCCGGGCAAG
301202j2 G44X, L45X, W47X: (SEQ ID NO. 46)
5′-GCGCCGGGCAAGNNSNNSGAGNNSGTCGCGGACGTG
301202j3 Y95 X: (SEQ ID NO. 47)
5′-ACCGCGGTCTACNNSTGCGCCCGGAAC
301202j4 W109X: (SEQ ID NO. 48)
5′-ACTTCGACTACNNSGGCCAGGGCACC
7887: (SEQ ID NO. 49)
5′-GAATGACTTGGTTGAGTACTCACCAGTCAC

(Thus changing the Mlul site found in the ampicillin resistance gene and used for cutting to a Scal site).

A library was made in E. coli using the plasmid pENI-herceptinl as template, the mutation primers 301202j1, 301202j2, 301202j3, 301202j4 and primer 7887 as selection primer along with the the commercial kit, Chameleon double-stranded, site-directed mutagenesis kit can be used according to the manufacturer's instructions (Stratagene).

The resulting E. coli library was transformed in the Aspergillus strain Jal355 as mentioned in patent WO 00/24883 A1. (See above)

The resulting transformants were screened as mentioned in patent WO 01/98484 A1.

### Example 11

### Construction of pENI3318

pENI2155 and pHercetin4 were both cut with BamHI and SqrAI.

Vector fragment of pENI2155 and 1300 bp fragment of pHerceptin 4 was isolated from agarose gel, and ligated, thus creating pENI3318.

### Example 12

### Screening for Increased Solubility and Production of Heavy Chain Variable Domain Expressed from pENI3318.

In order to improve expression and solubility of heavy chain variable domain, amino acid residues involved in the contact between the heavy chain and the light chain were mutated. Potentially any amino acid change could do so, by changing the overall protein structure slightly. The amino acids residues should preferably be mutated to hydrophilic residues, such as K, R, H, D, E, G, N, Q, C, S, T or Y.

The positions to be mutated should in the given example preferably be: V37, Q39, G44, L45, W47, Y95 and W109i.

In order to increase expression and solubility the following screen was performed: The following phosphorylated primers were designed, in which X designates naturally occurring amino acids and the amino acid positions refer to SEQ ID NO 1.

301202j1 V37X, Q39X: (SEQ ID NO 45)
5′-ACGATGGACTGGNNSCGGNNSGCGCCGGGCAAG
301202j2 G44X, L45X, W47X: (SEQ ID NO 46)
5′-GCGCCGGGCAAGNNSNNSGAGNNSGTCGCGGACGTG
301202j3 Y95 X: (SEQ ID NO 47)
5′-ACCGCGGTCTACNNSTGCGCCCGGAAC
301202j4 W109X: (SEQ ID NO 48)
5′-ACTTCGACTACNNSGGCCAGGGCACC
19670: (SEQ ID NO 50)
5′-CCCCATCCTTTAACTATAGCG
060302J1: (SEQ ID NO 51)
5′-AGAGCTTAAAGTATGTCCCTTG

A PCR was run using pENI3318 as template and the mutation oligoes 301202j1, 301202j2, 301202j3, 301202j4 and oligo19670 using Phusion as recommended by manufacture (Finnzymes).

The fragments (900 bp-1100 bp) were isolated from an agarose gel. Using the purified fragments and pENI3318 as template, with oligo 060302j1, a new PCR was run using Phusion. The resulting PCR fragment was cut BamHI and SgrAI and ligated into pENI2155 cut with the same enzymes.

The ligation was electrotransformed into XL10-gold giving 4500 coli clones, and non on the control ligation of the vector alone.

The resulting E. coli library was transformed in the Aspergillus strain Jal355 (as mentioned in patent WO 00/24883 A1).

JaL355 was transformed with library using standard procedures, cf., as described in WO 98/01470. The cells were then cultured on Cove plates at 37° C.

Transformants appeared after three days incubation at a transformation frequency of 10⁴-10⁵/μg DNA.

400 independent transformants were inoculated into a 96-well microtiter dish containing 200 μl YPM in each well. Aspergillus transformed with the parental plasmid pENI3318 were inoculated as triplicate for control.

After three days of incubation at 34° C., media from the cultures in the microtiter dish were assayed for lipase activity. A 5 μl aliquot of media from each well was added to a microtiter well containing 200 μl of a lipase substrate of 0.018% p-nitrophenylbutyrate, 0.1% Triton X-100, 10 mM CaCl₂, 50 mM Tris pH 7.5. Activity was assayed spectrophotometrically at 15-second intervals over a five minute period, using a kinetic microplate reader, using a standard enzymology protocol (e.g., Enzyme Kinetics, Paul C. Engel, ed., 1981, Chapman and Hall Ltd.). Briefly, product formation is measured during the initial rate of substrate turnover and is defined as the slope of the curve calculated from the absorbance at 405 nm every 15 seconds for 5 minutes. The 34 strains expressing the highest level of lipase were isolated. No lipase expression was seen from pENI3318 Aspergillus transformants. The increased lipase expression was taken as an indication of increased expression and solubility of the heavy chain variable domain. Plasmids were isolated from each transformant and mutations identified.

### Example 13

Screening for Increased Solubility and Production of Heavy Chain Variable Domain Expressed from pENI3318.

In order to improve expression and solubility of heavy chain variable domain amino acid residues involved in the contact between the heavy chain and the light chain were mutated. Potentially any amino acid change could do so, by changing the overall protein structure slightly. The amino acids residues should preferably be mutated to hydrophilic residues, such as K, R, H, D, E, G, N, Q, C, S, T or Y.

The positions to be mutated should in the given example preferably be: V37, Q39, G44, L45, W47, Y95 and W109.

In order to increase expression and solubility the following screen was performed The following phosphorylated primers were designed, in which X designates naturally occurring amino acids and the amino acid positions refer to SEQ ID NO 1.

301202j1 V37X, Q39X: (SEQ ID NO 45)
5′-ACGATGGACTGGNNSCGGNNSGCGCCGGGCAAG
301202j2 G44X, L45X, W47X: (SEQ ID NO 46)
5′-GCGCCGGGCAAGNNSNNSGAGNNSGTCGCGGACGTG
301202j3 Y95 X: (SEQ ID NO 47)
5′-ACCGCGGTCTACNNSTGCGCCCGGAAC
301202j4 W109X: (SEQ ID NO 48)
5′-ACTTCGACTACNNSGGCCAGGGCACC
7887: (SEQ ID NO 49)
5′-GAA TGA CTT GGT TGA GTA CTC ACC AGT CAC

(Thus changing the Mlu l site found in the ampicillin resistance gene and used for cutting to a Scal site).

A library was made in E. coli using the plasmid pENI3318 as template, the mutation oligoes 301202j1, 301202j2, 301202j3, 301202j4 and oligo7887 using the below mentioned procedure.

The resulting E. coli library was transformed in the Aspergillus strain Jal355 (as mentioned in patent WO 00/24883 A1).

JaL355 was transformed with library using standard procedures, cf., as described in WO 98101470. The cells were then cultured on Cove plates at 37° C.

Transformants appeared after three days incubation at a transformation frequency of 10⁴-10⁵/μg DNA.

40 independent transformants were inoculated into a 96-well microtiter dish containing 200 μl YPM in each well. Aspergillus transformed with the parental plasmid pENI3318 were inoculated as triplicate for control.

After three days of incubation at 34° C., media from the cultures in the microtiter dish were assayed for lipase activity. A 5 μl aliquot of media from each well was added to a microtiter well containing 200 μl of a lipase substrate of 0.018% p-nitrophenylbutyrate, 0.1% Triton X-100, 10 mM CaCl₂, 50 mM Tris pH 7.5. Activity was assayed spectrophotometrically at 15-second intervals over a five minute period, using a kinetic microplate reader, using a standard enzymology protocol (e.g., Enzyme Kinetics, Paul C. Engel, ed., 1981, Chapman and Hall Ltd.). Briefly, product formation is measured during the initial rate of substrate turnover and is defined as the slope of the curve calculated from the absorbance at 405 nm every 15 seconds for 5 minutes. The 8 strains expressing the highest level of lipase were isolated. The increased lipase-expression was taken as an indication of increased expression and solubility of the heavy chain variable domain. No lipase expression was seen from pENI3318 Aspergillus transformants. SDS-page was run and confirmed improved expression. Plasmids were isolated from each transformant and mutations identified. The following mutants were found—all produced in higher quantities than the wild type:

V37S,D,G

Q39C,W,S

L45G

W47G,R,L

Y95L,F

Mutagenesis method using Proof start polymerase (Qiagen, 202205) and Taq thermostable ligase (Biolabs 208L): Mix 5 μl Ligase buffer and 5 μl Proof start buffer.

Add 10 μl dNTP (2.5 mM), 2.5 μl ligase and 2.5 μl Proff start polymerase. Transfer 2.5 μl to each PCR reaction tube (on ice). Add 100 ng template DNA. Add 20 pmol of each primer. Fill with sterile water to a total of 10 μl.

Run PCR reaction over night: 98° C. 1 min 30 times (96° C. 1 min, 50° C. 1 min, 65° C. 15 min).

Add 1 μl Dpn1 to PCR reaction and mix gently. Incubate 2 hours at 37° C. Transform into DH10b (chemically competent). Add 200 μl LB and grow 1 hour at 37° C. in eppendorf tube. Plate on LB+AMP and incubate at 37 degrees. Make DNA prep of clones.

1-7. (canceled)

8. A method for producing a functional human immunoglobulin, wherein a human heavy chain immunoglobulin, devoid of any light chain, is expressed, comprising the steps of:
a) transforming a filamentous host cell with a recombinant construct encoding a modified human heavy chain immunoglobulin, wherein the modifications comprise one or more mutations in the region of the heavy chain protein involved in contact with the light chain;
b) culturing said filamentous host cell under conditions promoting expression of said modified human heavy chain immunoglobulin; and
c) recovering said modified human heavy chain immunoglobulin.

9. The method according to claim 8, wherein the filamentous host is an Aspergillus host.

10. The method according to claim 8, wherein the human heavy chain immunoglobulin comprise at least the variable region and the Fc-region recognised by the Fc receptor.

11. The method according to claim 8, wherein the human heavy chain immunoglobulin comprise at least the variable region.

12. The method according to claim 8, wherein the modifications comprise mutations in the region of the heavy chain protein involved in contact with the light chain, said mutations comprising mutations in either of the residues V37, Q39, G44, L45, W47, Y95 and W109 in SEQ ID NO 1, said sequence representing the heavy chain variable domain of the human immunoglobulin, Herceptin, or mutations in functionally equivalent residues in other human heavy chain immunoglobulins.

13. The method according to claim 8, wherein the modifications further comprise mutations in the region of the heavy chain immunoglobulin involved in contact with the antigen.

14. The method according to claim 9, wherein the modifications further comprise mutations in the region of the heavy chain immunoglobulin involved in contact with the antigen.

15. The method according to claim 10, wherein the modifications further comprise mutations in the region of the heavy chain immunoglobulin involved in contact with the antigen.

16. The method according to claim 11, wherein the modifications further comprise mutations in the region of the heavy chain immunoglobulin involved in contact with the antigen.

17. The method according to claim 12, wherein the modifications further comprise mutations in the region of the heavy chain immunoglobulin involved in contact with the antigen.

18. The method according to claim 8, wherein the modifications further comprise mutations in the region of the heavy chain immunoglobulin involved in contact with the antigen, said mutations comprising mutations in either of the residues comprised in the positions 27-35, 50-57 and 99-108 in SEQ ID NO 1, said sequence representing the heavy chain variable domain of the human immunoglobulin, Herceptin, or mutations in functionally equivalent residues in other human heavy chain immunoglobulins.

19. The method according to claim 9, wherein the modifications further comprise mutations in the region of the heavy chain immunoglobulin involved in contact with the antigen, said mutations comprising mutations in either of the residues comprised in the positions 27- 35, 50-57 and 99-108 in SEQ ID NO 1, said sequence representing the heavy chain variable domain of the human immunoglobulin, Herceptin, or mutations in functionally equivalent residues in other human heavy chain immunoglobulins.

20. The method according to claim 10, wherein the modifications further comprise mutations in the region of the heavy chain immunoglobulin involved in contact with the antigen, said mutations comprising mutations in either of the residues comprised in the positions 27-35, 50-57 and 99-108 in SEQ ID NO 1, said sequence representing the heavy chain variable domain of the human immunoglobulin, Herceptin, or mutations in functionally equivalent residues in other human heavy chain immunoglobulins.

21. The method according to claim 11, wherein the modifications further comprise mutations in the region of the heavy chain immunoglobulin involved in contact with the antigen, said mutations comprising mutations in either of the residues comprised in the positions 27-35, 50-57 and 99-108 in SEQ ID NO 1, said sequence representing the heavy chain variable domain of the human immunoglobulin, Herceptin, or mutations in functionally equivalent residues in other human heavy chain immunoglobulins.

22. The method according to claim 12, wherein the modifications further comprise mutations in the region of the heavy chain immunoglobulin involved in contact with the antigen, said mutations comprising mutations in either of the residues comprised in the positions 27-35, 50-57 and 99-108 in SEQ ID NO 1, said sequence representing the heavy chain variable domain of the human immunoglobulin, Herceptin, or mutations in functionally equivalent residues in other human heavy chain immunoglobulins.
